(19) United States
(12) Patent Application Publication
(10) Pub. No.: US 2002/0187066 A1
(43) Pub. Date: Dec. 12, 2002

(54) Apparatus and method using capillary discharge plasma shower for sterilizing and disinfecting articles

(75) Inventors: Dong Woo Yu, Demarest, NJ (US); Steven Kim, Harrington Park, NJ (US)

Correspondence Address: MORGAN LEWIS & BOCKIUS LLP, 1111 PENNSYLVANIA AVENUE NW, WASHINGTON, DC, 20004, US

(73) Assignee: SKION CORPORATION

(21) Appl. No.: 09/875,041

(22) Filed: Jun. 7, 2001

(51) Int. Cl. A61L 2/00; B01J 19/08

(52) U.S. Cl. 422/22; 422/23; 422/186.04; 422/186.05; 250/455.11

(57) ABSTRACT

An apparatus for sterilizing an article using capillary discharge atmospheric pressure plasma is disclosed. The apparatus includes a power supplier providing a potential to the apparatus, a plasma generating head, a gas supplier providing a sufficient amount of working gas to the plasma generating head, and a body including a handle and coupled to the plasma generating head and the gas supplier. The plasma generating head includes, a metal electrode receiving the potential, a dielectric having at least one capillary therein coupled to the metal electrode, and a shield body surrounding at least a portion of the metal electrode except for the capillary.

### BACKGROUND OF THE INVENTION

1. Field of the Invention

The present invention relates to an apparatus and method for disinfecting and sterilizing articles, and in particular the present invention relates to a method and system using a capillary discharge plasma shower for disinfecting and sterilizing articles.

2. Discussion of the Related Art

A number of sterilizing and disinfecting systems and methods have been developed to treat articles. Amongst them are steam sterilization, chemical sterilization, thermal inactivation, irradiation, etc.

In steam sterilization for example, the article is placed in an apparatus similar to an autoclave. In a typical steam sterilization process, the item is exposed to steam at approximately 250° F., for about 30 minutes.

Steam sterilization has been disadvantageous for sterilization for it requires relatively large and costly equipment. Furthermore, steam sterilization equipment is expensive to operate due in large part to the substantial heating requirements. Steam sterilization is also disadvantageous in that it generally does not eliminate highly objectionable visual evidence of the contamination.

Chemical sterilization usually makes use of soaps and detergents. However, the use of chemicals, especially in large quantities also has its drawbacks. Many of the chemicals used in the sterilization process are not biodegradable. Therefore, although chemical sterilization might be good disinfecting articles, their harm to the environment outweighs their benefits.

Thermal inactivation is accomplished by the application of dry heat in an oven, which is usually operated by electricity. This process involves heating the article with an electric source, and then maintaining a temperature of around 200° F. for approximately two hours in a large enclosed chamber. This process also tends to be expensive.

Irradiation exposes wastes to ultraviolet or ionizing radiation from a source such as cobalt 60 in an enclosed, shielded chamber. Disadvantages are the large initial cost of the equipment, and the skilled personnel required for safe operation just to name a few. Furthermore, this method is only effective if the ultraviolet radiation reaches the contaminant and generally little radiation penetrates the item, which tends not to be ultraviolet transparent.

### SUMMARY OF THE INVENTION

Accordingly, the present invention is directed to a method and an apparatus for plasma treatment using capillary discharge plasma shower that substantially obviate one or more of the problems due to limitations and disadvantages of the related art.

A feature of the present invention is to provide a method and system that is used in hospitals, medical centers, and treatment centers for both human and animals, to sterilize instruments used in surgical and medical procedures.

Another feature of the present invention is to provide a system and method that is used in the sterilization of air in medical areas where sterilization is fundamental in the prevention of the spread of disease by bacteria, germs, viruses and fungi.

A further feature of the present invention is to provide a system and method used for sterilization of medical equipment utilized in surgery, and medical devices that are employed in areas where sterilization is necessary, such as diagnostic equipment used in medicine.

Furthermore, the present invention provides a system and method for the sterilization of clothing (fabric, paper and disposable) masks, eyeglasses and eyewear, gloves, shoes, and the like. The present invention is also utilized in the sterilization of sheets, bed clothing, blankets and towels used in hospitals, medical centers and treatment centers.

The present invention can also be utilized in the sterilization of materials used in hospitals, medical centers and treatments centers, such as wound dressings, gauze, bandages, cotton swabs, suture materials and the like.

A further feature of the invention is to provide a method and system used for the sterilization of humans and animals, such as for wounds, cuts, or any type of infection caused by bacteria, virus or fungi. For example, a person suffering from athlete's foot can utilize the system and method of the present invention for treating the affected area.

Additional features and advantages of the invention will be set forth in part in the description that follows, and in part will be obvious from the description, or may be learned by practice of the invention. The features and advantages of the invention will be realized and attained by means of the elements and combinations particularly pointed out in the appended claims.

To achieve these and other advantages and in accordance with the purpose of the invention, as embodied and broadly described, the present invention provides a portable apparatus for sterilizing an article using a capillary discharge atmospheric pressure plasma, including a power supplier providing a potential to the apparatus, a plasma generating head generating the capillary discharge atmospheric plasma, wherein the plasma generating head includes, a metal electrode receiving the potential, a dielectric having at least one capillary therein coupled to the metal electrode, and a shield body surrounding at least a portion of the metal electrode except for the capillary; a gas supplier providing a sufficient amount of working gas to the plasma generating head; and a body including a handle and coupled to the plasma generating head and the gas supplier.

The present invention further provides a sterilizing chamber using a capillary discharge atmospheric pressure plasma for sterilizing articles comprising an enclosed chamber enclosing the articles, at least one plasma generating head placed in the chamber, wherein the plasma generating head generating the capillary discharge atmospheric pressure plasma includes a metal electrode receiving the potential, a dielectric having at least one capillary therein coupled to the metal electrode, and a shield body surrounding at least a portion of the metal electrode except for the capillary, and a gas supplier providing a sufficient amount of working gas to the plasma generating head.

Furthermore, the present invention provides a method for sterilizing articles using a capillary discharge atmospheric pressure plasma generating apparatus, the method comprising the steps of placing the apparatus in close proximity to the articles, wherein the apparatus comprises a power supplier providing a potential to the apparatus, a plasma generating head generating the capillary discharge atmospheric pressure plasma, wherein the plasma generating head comprises a metal electrode receiving the potential, a dielectric having at least one capillary therein coupled to the metal electrode, and a shield body surrounding at least a portion of the metal electrode except for the capillary, a gas supplier providing a sufficient amount of working gas to the plasma generating head, and a body including a handle and coupled to the plasma generating head and the gas supplier, applying the potential to the metal electrode, generating the capillary discharge atmospheric pressure plasma from the capillary to sterilize the articles, and relocating the plasma generating head with respect to the articles if necessary.

Additionally, the present invention provides a method for sterilizing an article using a capillary discharge atmospheric pressure plasma generating chamber, the method comprising the steps of placing the articles in the chamber, wherein the chamber comprises an enclosed chamber enclosing the article, a power supplier providing a potential to the chamber, at least one plasma generating head generating the capillary discharge atmospheric plasma, wherein the plasma generating head comprises a metal electrode receiving the potential, a dielectric having at least one capillary therein except for the capillary, a gas supplier providing a sufficient amount of working gas to the plasma generating head, and a body including a handle and coupled to the plasma generating head and the gas supplier, applying the potential to the metal electrode, generating the capillary discharge atmospheric pressure plasma from the capillary to sterilize the article, and relocating the article with respect to the plasma generating head if necessary.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute part of this specification, illustrate embodiments of the invention, and together with the description serve to explain the principles of the invention.

In the drawings:

FIG. 1 shows an individual using a portable capillary discharge plasma device disinfecting several articles of the present invention.

FIG. 2 shows an individual in a disinfecting chamber of the present invention.

FIG. 3 shows a capillary discharge plasma head of the present invention.

FIG. 4 shows a cross-sectional view of a capillary discharge plasma head of a first embodiment in the present invention.

FIG. 5 shows a cross-sectional view of a capillary discharge plasma head of a second embodiment in the present invention.

FIG. 6 shows a cross-sectional view of a capillary discharge plasma head of a third embodiment in the present invention.

### DETAILED DESCRIPTION OF THE PREFERRED EMBODIMENTS

Reference will now be made in detail to the preferred embodiments of the present invention, examples of which are illustrated in the accompanying drawings. Wherever possible, the same reference numbers will be used throughout the drawing to refer to the same of like parts. The present invention provides an apparatus and method for sterilizing a plurality of articles with the use of a capillary plasma discharge. The plasma discharge apparatus may be designed in the form of a hand held or stationary device wherein the plasma discharged from a capillary is focused on the article. The plasma discharge can destroy bacteria, germs, viruses and fungi. The use of the plasma discharge is not limited to articles, but can also be used on living creatures such as humans and animals. For example, the plasma discharge could be directed onto a person's foot to destroy athlete's foot, caused by fungi. The plasma discharge also finds use in a variety of locations, such as hospitals, medical centers, and treatment centers, for both humans and animals. Furthermore, the present invention could be used to sterilize instruments used in surgical and medical procedures.

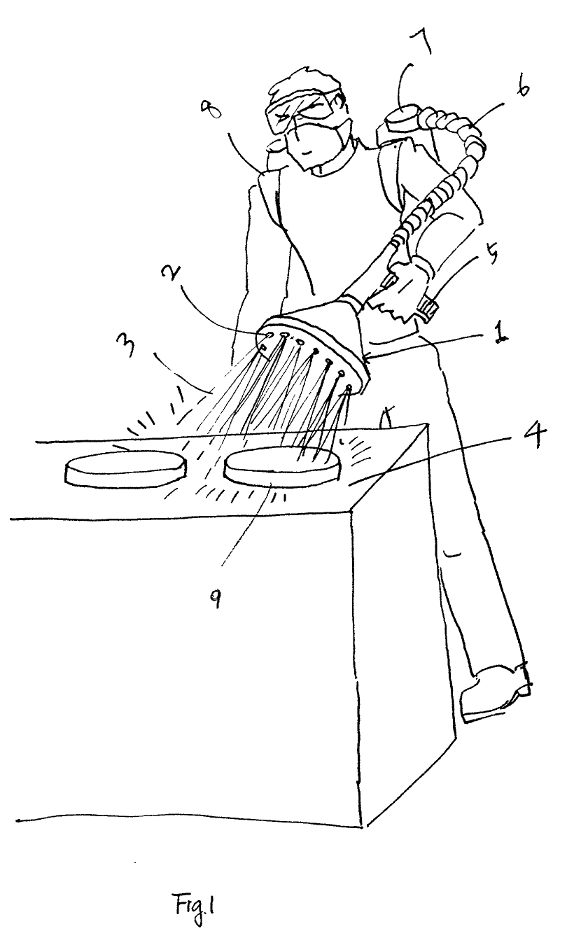

FIG. 1 shows a portable capillary plasma discharge device operated by an individual. The device can be mounted on the individual's back similarly to a backpack (8). The individual must wear protective gear such as a special suit, footwear and a mask to avoid a possible infection from the contaminated article while operating the machine.

FIG. 1 also illustrates several articles (9) being sterilized. The articles can be placed on a special table top or counter top (4) where the sterilization is to take place. The sterilization device (1) comprises a plasma head (2), a handle (5), a tube (6) and a gas tank (7). The individual holds the device by the handle (5) and aims at the article to be sterilized. After depressing a trigger (not shown), plasma (3) is discharged from the device sterilizing the article.

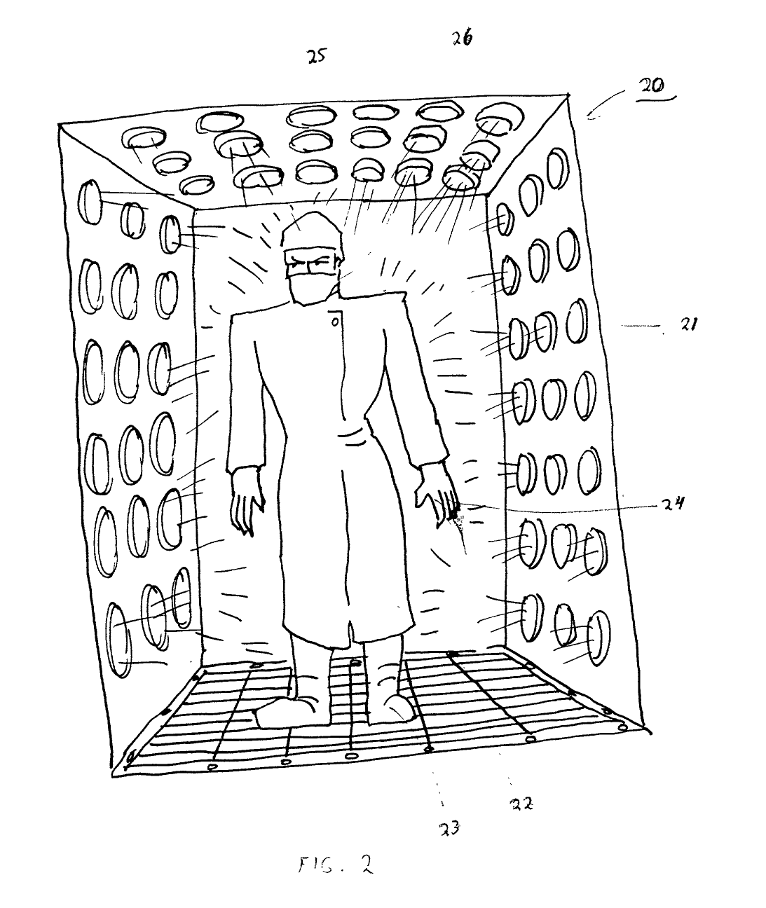

FIG. 2 illustrates a sterilization chamber (20). The sterilization chamber generally comprises a plurality of plasma discharge showerheads (21) arranged on the walls and on the floor of the chamber. An individual wearing clothes that need to be disinfected or sterilized such as prior to an operation or immediately thereafter, enters the chamber. For example, the individual would be wearing a mask (26), head cap (25), suit (22), footwear (23), and gloves (24). Once the individual enters the chamber, the plasma showerheads are activated and the sterilization process of the clothing begins. All of the clothes and footwear of the individual are exposed to the plasma shower, and the articles become disinfected. After a defined amount of time, the showerheads are turned off and the garments worn by the individual are sterilized.

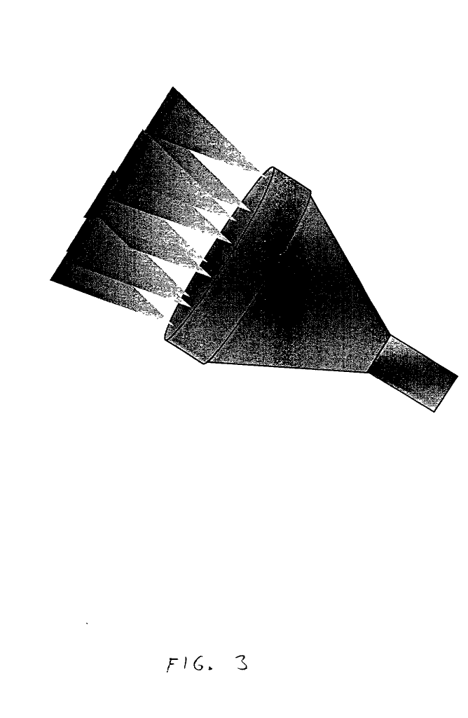

FIG. 3 illustrates a plasma showerhead. The plasma showerhead comprises a plurality of openings through which the plasma exits the device.

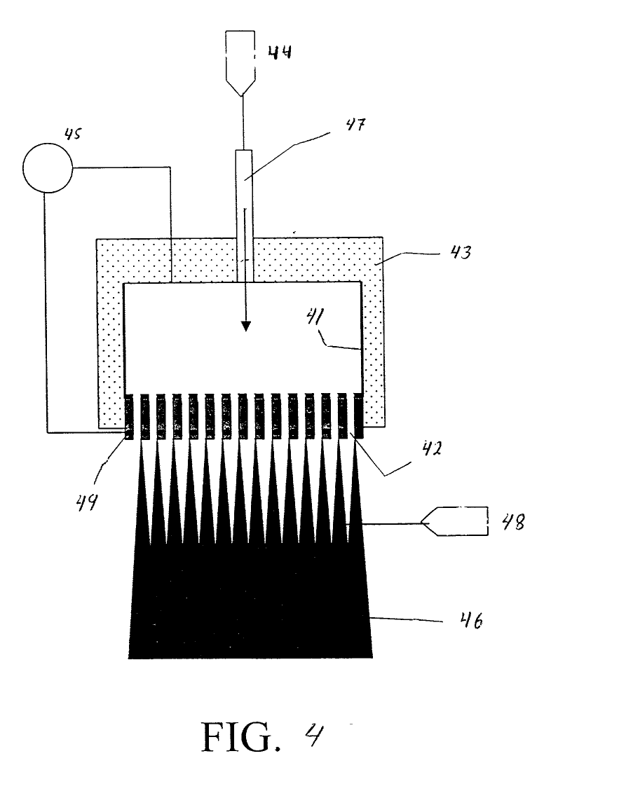

FIG. 4 is a schematic cross-sectional view illustrating an apparatus for plasma treatment using a capillary plasma shower according to a first embodiment of the present invention. As shown in FIG. 4, an apparatus for plasma treatment using a capillary plasma shower according to a first embodiment includes a first metal electrode (41), a capillary dielectric (42), a shield body (43), a gas supplier (44), a power supply (45) and a gas tube (47).

The first metal electrode (41) is coupled to the power supply (45). Either a DC or a RF potential may be applied to the first metal electrode (41). In the case where the RF potential is applied, it is preferably in the range of 10 kHz to 200 MHz.

The capillary dielectric (42) has first and second sides and coupled to the first metal electrode (41) through the first side of the capillary dielectric (42). The capillary dielectric (42) has at least one capillary. For example, the number of capillaries may range from one to thousands. A thickness of the capillary dielectric (42) may be in the range of 2 mm to 300 mm. A diameter of each capillary is preferably in the range of 200 μm to 30 mm.

The first metal electrode (41) can be in the form of a metal cylinder or a parallelepiped having one or more holes in the bottom surface that are substantially aligned with capillaries in the capillary dielectric (42). One side of the capillary dielectric (42) is coupled to the first metal electrode (41) inside the shield body (43) while another side of the capillary dielectric (42) is outside the shield body (43) and exposed to a article.

The shield body (43) surrounds the first metal electrode (41) and the capillary dielectric (42), so that it prevents unnecessary area from generating discharge. The shield body (43) is made of a dielectric material. A grip may be formed on the shield body (43) so that a user can conveniently hold it. The gas supplied with the metal electrode (41) passes through the capillary. Since a high electric field is maintained across the capillary dielectric (42), a high-density discharge beam is generated in the capillary. The gas may be a carrier gas or a reactive gas depending upon a specific application of the apparatus. An additional gas supplier (48) may be supplied to a space between the capillary dielectric (42) and an article to be treated by plasma discharge.

The article to be treated by the apparatus for plasma treatment using the capillary plasma shower discharge may act as a counter electrode.

The gas tube (47) made of a metal or a dielectric material is further coupled to a metal electrode (41), so that gas is supplied by the gas supplier (44) through the gas tube (47). The gas can be any gas, preferably, it can be Ar, He, O₂ or air, or any mixture of these gases.

A second metal electrode (49) can be mounted on the second side of the capillary dielectric (42). Preferably, the second metal electrode (49) is completely encapsulated in the capillary dielectric to prevent arcing between the electrodes. This second metal electrode (49) can be used to provide additional focusing of the plasma discharge (46).

The second metal electrode (49) is connected to the power supply (45) in series with the first metal electrode (41). This provides a potential difference with respect to the first metal electrode (41). It is unnecessary to connect the article (not shown) to ground and articles made of virtually any kind of material, such as metal, ceramic, and plastic, can be treated by the apparatus of the present invention.

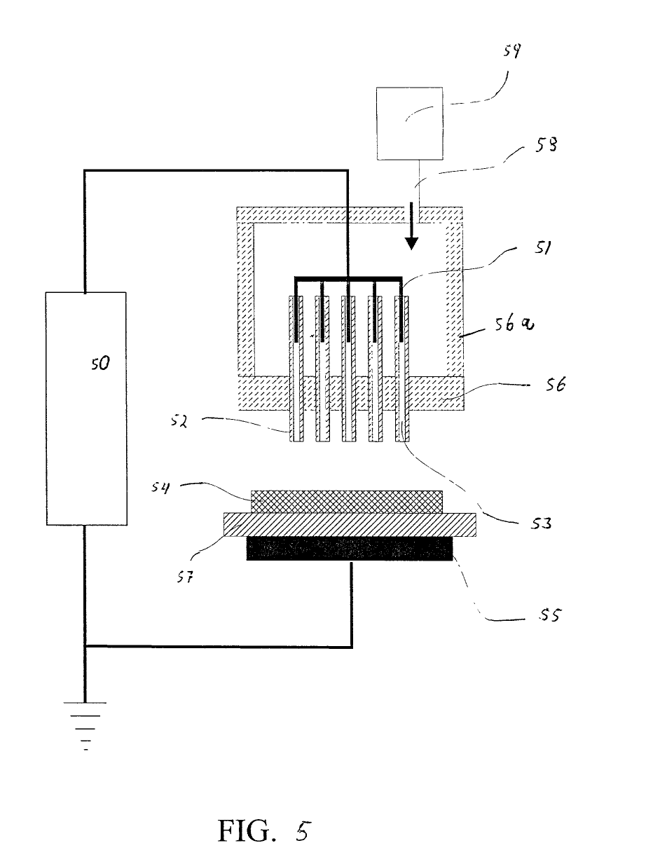

FIG. 5 is a schematic cross-sectional view illustrating an apparatus for plasma treatment using a capillary plasma shower according to a second embodiment of the present invention. As shown in FIG. 5, an apparatus for plasma treatment using a capillary plasma shower according to a second embodiment comprises a power supply (50), pin electrodes (51), capillaries (52), dielectric body (56), and chamber (56a). Additionally, the apparatus comprises a counter electrode (55) electrically coupled to the pin electrodes, a gas supplier (59), and a gas tube (58).

One of the terminals of the power supply (50) is coupled to pin electrodes (51), while the other terminal is coupled to the counter electrode (55) and is grounded. Either a DC or a RF potential may be applied to the pin electrode (51). In the case where the RF potential is applied, it is preferably in the range of 10 kHz to 200 MHz.

The dielectric body (56) has first and second sides, the first side coupled to the pin electrodes (51), and the second side having at least one capillary that extends into a portion of the dielectric body (56). The pin electrodes (51) and the capillaries (52) are substantially aligned and generally have a one to one correspondence. Although there are no critical limitation in a thickness of the dielectric body (56), the thickness of the dielectric body (56) may be in the range of 1 mm to 3 cm. A diameter of each capillary is preferably in the range of 0.2 mm to 0.8 mm. A non-conductive substrate (54) is placed between pin electrodes (51) and counter electrode (55), and subjected to a plasma treatment using capillary discharge plasma.

The pin electrodes (51), and a portion of the capillaries, are enclosed in the chamber (56a). Chamber (56a) additionally comprises a gas tube (58), and a gas supplier (59). The gas tube (58) is made of a metal or a dielectric material and is connected to gas supplier (59). The gas supplied to chamber (56a) can be any gas, preferably it can be Ar, He, O₂ or air, or any mixture of these gases.

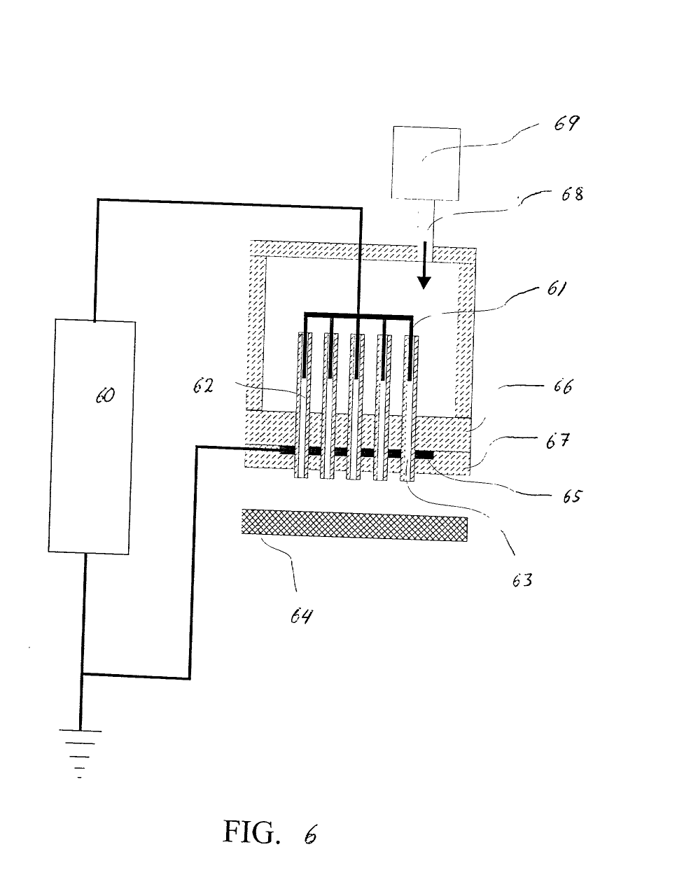

FIG. 6 illustrates a cross-sectional view of a plasma showerhead of a third embodiment. Similarly to the previous embodiment, the apparatus includes at least one pin electrode (61) for receiving a power source (60). A dielectric body (67) having first and second sides, wherein the first side is coupled to the pin electrode (61) and the second side has at least one capillary (62) extending to a direction of the first side of the dielectric body. Each capillary is substantially aligned with each pin electrode, and a counter electrode electrically coupled to the pin electrode for generating the plasma from each capillary. The apparatus further includes a gas supplier (69) and a gas tube (68) extending into a chamber (66a) housing pin electrodes (61).

In order to demonstrate a feasibility of practical applications in industries, experiments were conducted using an apparatus and method as previously discussed in the present invention. As recommended in the procedure of the AOAC (Official Method Analysis of the Association of Official Analytical Chemists, 12^{th} Ed. November 1975), Bacillus subtillis and Bacillius stearothermophilus were used in the experiments. Control spore strips (American Sterilizer Co. SPORIDI®) made of Bacillus subtillis and Bacillius stearothermophilus were tested under different conditions: ETOC (ethylene oxide certified) method, DHC (dry heat certified) method, and CDAP (capillary discharge atmospheric plasma) method.

First, the number of survivor for Bacillus subtillis after treated by the CDAP method was measured from 0 second to 120 seconds. Before the CDAP treatment, the number of survivor of Bacillus subtillis was about 950,000. The numbers were significantly reduced to about 600,000 in 60 seconds and about 200,000 in 120 seconds after the CDAP treatment.

D-value was also measured for the ETOC, DHC, and CDAP methods. D-value is described as the time necessary to reduce the population of cells by one log or 90%. These values are determined from the plots of the number of survivors vs. time. Thus, based on the data, D-value is calculated for each method. For the ETOC and DHC methods, D-values were about 3.9 minutes and 1.5 minutes, respectively. D-value for the CDP method of the present invention was 2.95 minutes. The D-value of the present invention was higher than that of the DHC method. However, the DHC method has some disadvantages in application. For example, the DHC method cannot directly be applied to a living human body or any animal because of hot and dry conditions.

On the other hand, the CDAP treatment has almost no restriction in applying because it's non-thermal nature of plasma.

Similar data were obtained from Bacillius stearothermophilus in the number of survivor and D-value. Before the CDP treatment, the number of survivor for Bacillus subtillis was about 4,200,000. The numbers were also significantly reduced to about 1,000,000 in 60 seconds and about 240,000 in 120 seconds after the CDAP treatment. For Bacillius stearothermophilus, D-value obtained for the sample treated by the CDAP method was lower than that by the DHC method. D-values for the CDAP and DHC method were about 1.54 and 1.90 minutes, respectively.

Accordingly, the experimental results indicate that the CDAP method of the present invention is very effective in sterilizing Bacillius stearothermophilus.

While the invention has been described in detail and with reference to specific embodiments thereof, it will be apparent to one skilled in the art that various changes and modifications can be made therein without departing from the spirit or scope thereof. Thus, it is intended that the present invention covers the modifications and variations of this invention provided they come within the scope of the appended claims and their equivalents.

What is claimed is:

1. A portable apparatus for sterilizing an article using a capillary discharge atmospheric pressure plasma, comprising:
a power supplier providing a potential to the apparatus;
a plasma generating head generating the capillary discharge atmospheric plasma, wherein the plasma generating head includes,
a metal electrode receiving the potential,
a dielectric having at least one capillary therein coupled to the metal electrode, and
a shield body surrounding at least a portion of the metal electrode except for the capillary;
a gas supplier providing a sufficient amount of working gas to the plasma generating head; and
a body including a handle and coupled to the plasma generating head and the gas supplier.

2. The apparatus according to claim 1, wherein the dielectric has a thickness in the range of 2 mm to 300 mm.

3. The apparatus according to claim 1, wherein the at least one capillary has a diameter in the range of 200 μm to 30 mm.

4. The apparatus according to claim 1, wherein the potential provided to the apparatus is a DC or a RF potential.

5. The apparatus according to claim 4, wherein the RF potential is in the range of 10 KHz to 200 MHz.

6. A sterilizing chamber using a capillary discharge atmospheric pressure plasma for sterilizing articles, comprising:
an enclosed chamber enclosing the articles;
a power supplier providing a potential to the chamber;
at least one plasma generating head placed in the chamber, wherein the plasma generating head generating the capillary discharge atmospheric pressure plasma includes:
a metal electrode receiving the potential,
a dielectric having at least one capillary therein coupled to the metal electrode, and
a shield body surrounding at least a portion of the metal electrode except for the capillary; and
a gas supplier providing a sufficient amount of working gas to the plasma generating head.

7. The plasma generating head of claim 6, wherein the dielectric has a thickness in the range of 2 mm to 300 mm.

8. The plasma generating head of claim 6, wherein the at least one capillary has a diameter in the range of 200 μm to 30 mm.

9. The plasma generating head according to claim 6, wherein the potential provided to the chamber is a DC or a RF potential.

10. The plasma generating head according to claim 9, wherein the RF potential is in the range of 10 kHz to 200 MHz.

11. A method for sterilizing articles using a capillary discharge atmospheric pressure plasma generating apparatus, the method comprising the steps of:
placing the apparatus in close proximity to the articles, wherein the apparatus comprises a power supplier providing a potential to the apparatus, a plasma generating head generating the capillary discharge atmospheric pressure plasma, wherein the plasma generating head comprises a metal electrode receiving the potential, a dielectric having at least one capillary therein coupled to the metal electrode, and a shield body surrounding at least a portion of the metal electrode except for the capillary, a gas supplier providing a sufficient amount of working gas to the plasma generating head, and a body including a handle and coupled to the plasma generating head and the gas supplier;
applying the potential to the metal electrode;
generating the capillary discharge atmospheric pressure plasma from the capillary to sterilize the articles; and
relocating the plasma generating head with respect to the articles if necessary.

12. The method according to claim 11, wherein the articles include space in medical areas, skin of humans and animals, containers and glassware for holding medicine, vaccines, injectables, pills, and any medical products for liquids, powders, and solids, and bulk chemicals, and containment of medicines in processing and after packaging.

13. A method for sterilizing an article using a capillary discharge atmospheric pressure plasma generating chamber, the method comprising the steps of:
placing the articles in the chamber, wherein the chamber comprises an enclosed chamber enclosing the article, a power supplier providing a potential to the chamber, at least one plasma generating head generating the capillary discharge atmospheric plasma, the plasma generating head comprising a metal electrode receiving the potential, a dielectric having at least one capillary therein except for the capillary, a gas supplier providing a sufficient amount of working gas to the plasma generating head, and a body including a handle and coupled to the plasma generating head and the gas supplier;
applying the potential to the metal electrode;
generating the capillary discharge atmospheric pressure plasma from the capillary to sterilize the article; and
relocating the article with respect to the plasma generating head if necessary.

14. The method according to claim 13, wherein the articles include space in medical areas, skin of humans and animals, containers and glassware for holding medicine, vaccines, injectables, pills, and any medical products for liquids, powders, and solids, and bulk chemicals, and containment of medicines in processing and after packaging.
